(19) United States
(12) Patent Application Publication
(10) Pub. No.: US 2014/0031944 A1
(43) Pub. Date: Jan. 30, 2014

(54) CYLINDRICAL GRAFT AND METHOD FOR PREPARING A RECIPIENT SITE AND IMPLANTING A CYLINDRICAL GRAFT INTO ALVEOLAR JAW BONE

(71) Applicants: Yoh Sawatari, Coral Gables, FL (US); Michael Peleg, Aventura, FL (US)

(72) Inventors: Yoh Sawatari, Coral Gables, FL (US); Michael Peleg, Aventura, FL (US)

(21) Appl. No.: 14/047,265

(22) Filed: Oct. 7, 2013

Related U.S. Application Data
(63) Continuation-in-part of application No. 12/920,633, filed on Sep. 2, 2010, now Pat. No. 8,574,236.
(60) Provisional application No. 61/064,651, filed on Mar. 18, 2008.

(51) Int. Cl. A61F 2/28

(52) U.S. Cl. CPC A61F 2/2803
USPC 623/17.17

(57) ABSTRACT

A method of fixating a graft to an area of deficient bone in the jaws comprising: exposing the area of deficient bone by incising and retracting soft tissue; selecting a drill bit having a diameter corresponding to the area of deficient bone to receive a graft; contacting the selected drill bit with the exposed deficient bone to form a generally cylindrical seat transversely into the side of the deficient bone for receiving the graft; selecting a cylindrical graft having a length and circular cross-sectional corresponding to the diameter and depth of the formed seat; fixating the graft to the formed seat with at least one screw; and closing the soft tissue to cover the previously exposed deficient bone.

This patent application is a Continuation-In-Part (CIP) patent application of co-pending patent application Ser. No. 12/920,633 filed on Sep. 2, 2010 which is a National Stage filing under 35 USC §371 of PCT International Patent Application No. PCT/US2009/034617 filed on Feb. 20, 2009 which was based on U.S. Provisional Patent Application No. 61/064,651 filed Mar. 18, 2008.

### BACKGROUND OF THE INVENTION

1. Field of the Invention

This invention relates to implanting a graft into the jaw bone and, more particularly, to a cylindrical graft and a method for preparing a recipient site of the jaw bone for receiving the cylindrical graft without the need to shape and contour the graft during the implant procedure.

2. Discussion of the Related Art

The use of allogeneic grafting has become a standard methodology to increase bone volume in deficient areas of the maxilla and mandible. The purpose of this grafting procedure is to prepare the patient for the eventual restoration of the patient's function and aesthetics with dental implants.

Conventionally, this procedure involves the surgical exposure of an area of deficient bone in the jaws, removal of a section of freeze dried bone from a tissue bank, contouring the section of freeze dried bone to follow the anatomical curvature of the target area of the jaw in which it is to be implanted and adapting the section of bone to passively rest on the jaw with maximum contact with the existing bone matter. The section of allogeneic bone is then fixated to the deficient jaw for approximately four months to allow fusion of the allogeneic bone to the jaw and eventual replacement with autologous bone.

As described, the process is routine yet difficult. The most time consuming and critical aspect of the procedure involves the adaptation of freeze dried bone to a deficient mandible. If there is poor adaptation, insufficient area contact, and/or a lack of stability at the time of fixation between the graft and the recipient jaw, the graft will fail to fuse and the patient will not receive any benefit from the graft procedure. Conventionally, the process which is utilized in the operating room or clinic involves the use of several different carbide burs to contour the surface which interfaces with the native jaw. Thus, once the surgical exposure is completed, the surgeon visualizes the recipient's site and through memory and repetitive test insert procedures, the surgeon will endeavor to produce the negative equivalent of the recipient site jaw contour in the allogeneic section. More specifically, the section of iliac crest is stabilized by the surgeon's hand or with an instrument while a rotary instrument on a handpiece is used to remove minimal amounts of bone to follow given contours of the jaw.

### BRIEF DESCRIPTION OF THE INVENTION

To address the inherent difficulty in shaping or sculpting the section of freeze dried bone to approximate the shape of the recipient site, the invention proposes a bone grafting procedure wherein the recipient site in the native jaw is manipulated to receive a cylindrical graft of specific length and diameter rather than having to contour the bone graft.

Thus, the invention is embodied in a method of fixating a cylindrical graft to an area of deficient bone in the jaws comprising: exposing the area of deficient bone by incising and retracting soft tissue; selecting a drill bit having a diameter corresponding to the area of deficient bone to receive the cylindrical graft; contacting the selected drill bit with the exposed deficient bone to form a generally cylindrical seat transversely into the area of deficient bone for receiving the correspondingly sized cylindrical graft; selecting the cylindrical graft that corresponds to the size of the formed seat; fixating the cylindrical graft to the formed seat with at least one screw; and closing the soft tissue to cover the previously exposed deficient bone.

According to the present invention, the cylindrical graft may be shaped from a variety of materials, including allogeneic bone, as well as alloplastic and xenographic grafting substances. Examples of alloplasts include, but are not limited to: porous fluorohydroxyapatitic (FHA); hydroxyapatite-calcium phosphate; calcium phosphate (CaP); betatricalcium phosphate (b-TCP); hydroxyapatite ceramic; HTR (hard tissue replacement polymer); bioglass; tricalcium phosphate; ceramic; and titanium. Examples of xenographs include, but are not limited to: bovine; coral; and algae.

### BRIEF DESCRIPTION OF THE DRAWINGS

For a fuller understanding of the nature of the present invention, reference should be made to the following detailed description taken in conjunction with the accompanying drawings in which:

FIG. 1 is a schematic perspective view of a drill guide that may be provided in an example embodiment of the invention for aligning a drill guide insert with a target area of natural jaw;

FIG. 2 is a schematic illustration of drill guide inserts corresponding to grafts of predetermined fixed diameter for manipulating the recipient site to conform to the size (i.e., diameter and length) of the cylindrical graft to be implanted;

FIG. 3 is a schematic perspective view of an example recipient site manipulated to have a cylindrical shape and size corresponding to that of the cylindrical graft; and

FIG. 4 is a schematic illustration of the patient's recipient site with the cylindrical section fixed in place, before soft tissue closure.

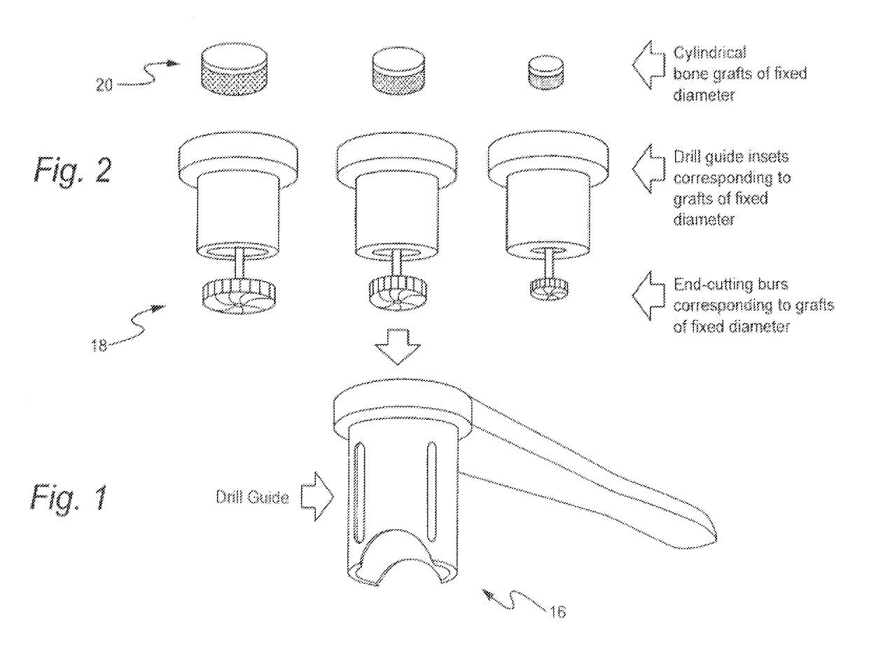

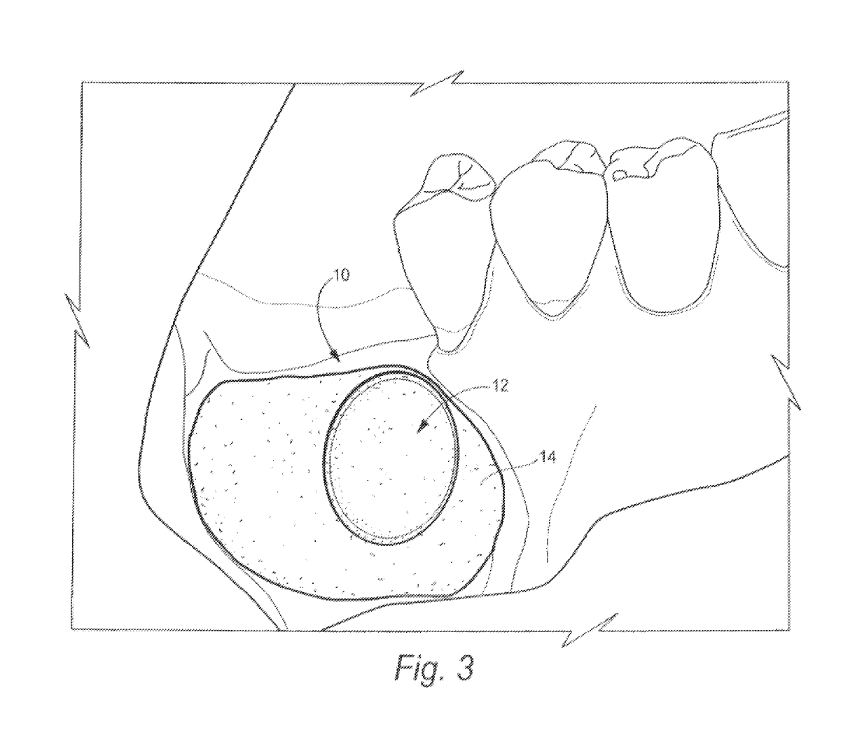

Like reference numerals refer to like parts throughout the several views of the drawings.

### DETAILED DESCRIPTION OF THE PREFERRED EMBODIMENT

To address difficulties of contouring a section of freeze dried bone for placement in a recipient site, as done in the conventional procedure, the patient recipient site in the native jaw is manipulated to have a shape corresponding to that of the cylindrical graft of the present invention.

In an example embodiment of the invention, the manipulation of the recipient site 10 involves the preparation of a cylindrical docking area 12 in which a cylindrical graft of specified diameter can be placed. Recipient sites may be of varying size. Therefore, a plurality of graft diameters can be contemplated and available for the procedure. For example, the diameter of the cylindrical graft may range from about 3 mm to 20 mm. Moreover, the height of thickness of the cylindrical graft may range from about 2 mm to 10 mm. The graft material can be allogeneic bone, an alloplastic substance or a xenographic substance. Based on an evaluation of the recipient site, a suitable diameter and length of the cylindrical graft can be selected and the preparation of the recipient site can be completed using a circular ended cutting burr of a fixed and known diameter. The proposed circular bur has a flat end cutting surface with peripheral flutes that resemble a starfish. Since the preparation of the cylindrical docking area does not need to be more than 1 to 2 mm deep, flutes or a distinct tapered leading edge is not necessary for the drill bit, and a circular bur as aforementioned may be used.

As will be appreciated, a grafting procedure utilizing cylindrical bone grafts for jaw volume augmentation will become far more efficient where the recipient sites can be formed to be of corresponding size (i.e., length and diameter) for congruent receipt of the cylindrical graft. Moreover, by standardizing the recipient site to one of a predetermined number of prescribed diameters, the need for time consuming manipulation, shaping and sizing of iliac crest in order to maximize recipient jaw contact is eliminated.

In the practice of the invention, according to an example embodiment, once the surgical site is evaluated, a standardized bur 18 is utilized to prepare the circular imprint 12 on the jaw bone 14. In an example embodiment of the invention, a surgical hand guide 16 is utilized to stabilize the circular bur 18 during the cut and to assist in giving the recipient site the appropriate angle to maximize contact and to determine the appropriate size. As will be appreciated, the drill guide is desirably used since otherwise, if a rotating flat base bur with small flutes were to contact a hard surface, the bur would spin out of position. Using the drill guide, the drill bit is contacted with the side of the exposed area of deficient jaw bone. The drill bit (e.g., bur) 18 is operatively rotated to form a cylindrical seat transversely into the side of the deficient jaw bone for receiving the graft.

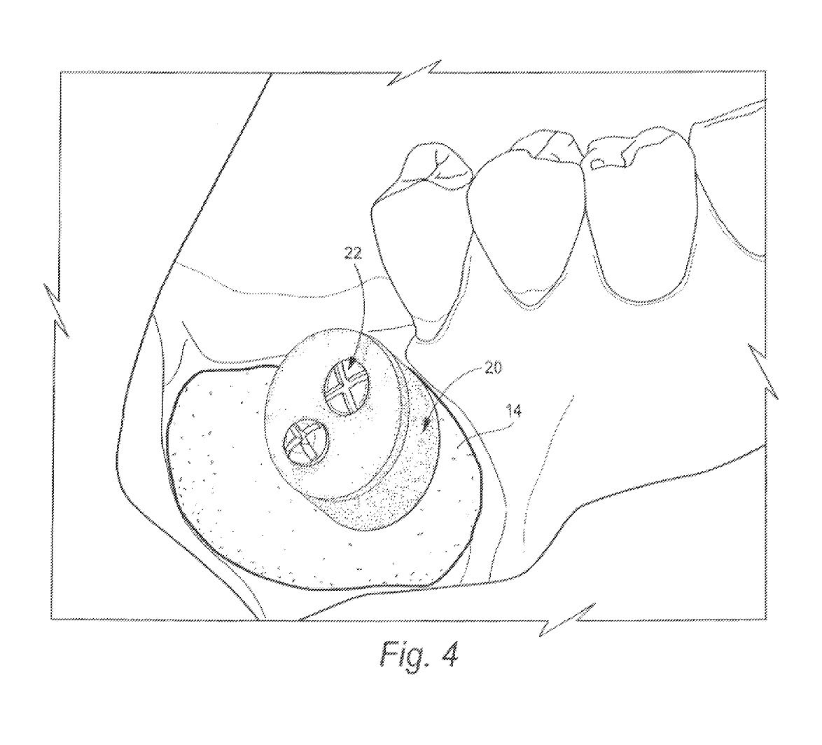

Once the seat is formed, the surgeon can then select the desired size cylindrical graft 20 from a given stock which matches the diameter and height required to appropriately reconstruct the area which is deficient. Since the recipient site 12 will be a certain diameter and surface contour, a cylindrical graft can be selected with that same diameter to fit in the prepared site. Once inserted, the cylindrical graft is fixated with screws 22 as shown in FIG. 4, followed by soft tissue closure and the procedure is complete. As will be appreciated, desirable compression of bone from the graft to the native jaw will come from the screw fixation. As currently proposed, the bone grafting procedure utilizes 1.5 mm by 5 mm to 18 mm screws with associated drill bit for fixation of the bone graft.

The graft of the invention is formed as a cylindrical block having opposite ends, an overall length defined between the opposite ends and a uniform transverse cross-sectional diameter throughout the entire length. The graft may be formed of one or more materials, such as allogeneic bone, alloplastic or xenographic grafting substances. Examples of alloplasts that may be used for the cylindrical bone graft of the present invention include, but are not limited to: porous fluorohydroxyapatitic (FHA); hydroxyapatite-calcium phosphate; calcium phosphate (CaP); betatricalcium phosphate (b-TCP); hydroxyapatite ceramic; HTR (hard tissue replacement polymer); bioglass; tricalcium phosphate; ceramic; and titanium. Examples of xenographs that may be used in the manufacture of the cylindrical bone graft of the present invention include, but are not limited to: bovine; coral; and algae.

As will be appreciated, the new procedure eliminates the need for any interface manipulation of the section of allogeneic bone as done in the conventional procedure, thus reducing the surgical time a patient must sustain. The reduction in surgical time would generally be expected to lead to a reduction in post-operative complications and morbidity associated with surgical procedures of the jaw.

In addition to the efficiency of the procedure, due to the standardization of multiple aspects of the grafting procedure, grafting using cylindrical grafts will appeal to more dental practitioners who may have been reluctant to complete the procedure in the past due to its complexity.

The invention may also be embodied in a surgical kit including circular burs or drill bits 18 of varying diameter for recipient site preparation, a surgical hand guide 16, and multiple units of cylindrical bone grafts 20 with diameters to match the diameters of the respective recipient sites to be formed with the circular drill bits. As noted above, screws 22 and an associated drill bit for fixation of the bone graft are used to complete the procedure. Thus, the kit may also distribute such screws 22 and the associated drill bit (not shown).

Drill guides of various designs are commonly used in maxillofacial fixation systems, e.g., for guiding drills for plate fixation. Such drill guides are conventionally provided for elongated fluted drill bits and to be engaged with the holes in the fixation plates to be secured in place, whereas the drill guide provided in accordance with an example embodiments of the invention has a through bore to accommodate the circular burr 18 and a distal end adapted to engage the natural bone of the jaw 14 rather than engage the bore of a plate.

Although circular burs are illustrated in FIG. 2 according to an example embodiment of the invention, as an alternative, a power-driven osteotome tool or other drill set may be used to form the recipient site, particularly if the graft is to be disposed in a deeper bore. A drill set for forming a bore of prescribed shape and diameter is disclosed for example in U.S. Pat. No. 7,247,020. It will be appreciated that a drill set of the type disclosed in '020 patent or a sub-set thereof may be used to form a suitable recipient site shape corresponding to the size of the cylindrical graft. In addition, or in the alternative, a combination of drill bits and expanders may be used to form a recipient site of suitable diameter and depth to accommodate the cylindrical graft. Reference is made in this regard to U.S. Pat. Nos. 6,899,715 and 7,241,144, the disclosures of each of which are incorporated herein by this reference.

While the invention has been described in connection with what is presently considered to be the most practical and preferred embodiment, it is to be understood that the invention is not to be limited to the disclosed embodiment, but on the contrary, is intended to cover various modifications and equivalent arrangements included within the spirit and scope of the appended claims.

What is claimed is:

1. A method of fixating a graft to an area of deficient bone in the jaws comprising:
exposing the area of deficient bone by incising and retracting soft tissue;
selecting a drill bit comprising a circular bur with a flat face cutting surface and having a diameter corresponding to the area of deficient bone to receive the graft;
selecting a drill guide having a through bore corresponding to the diameter of the selected drill bit;
directing the selected drill bit through the bore of the drill guide;
engaging a distal end of the drill guide against a side of the exposed area of deficient bone;
contacting the selected drill bit with the side of the exposed area of deficient bone and operating rotation of the selected drill bit to form a generally cylindrical seat transversely into the side of the deficient bone for receiving the graft, and said formed seat having a circular diameter and depth;
providing a plurality of cylindrical grafts of various cross-sectional diameter and length;
selecting one of said plurality of cylindrical grafts that has a cylindrical shape, cross-sectional diameter and length corresponding to the diameter and depth of said formed seat;
fixating the selected cylindrical graft to the formed seat with at least one screw; and closing the soft tissue to cover the previously exposed area of deficient bone.

2. A method of fixating a graft as in claim 1, wherein the cylindrical seat has a depth of 1 to 2 mm.

3. A method of fixating a graft as in claim 1, wherein said step of selecting said one of said plurality of cylindrical grafts comprises selecting a cylindrical graft formed of a material taken from the group comprising: allogeneic bone; one or more alloplastic substances; and one or more xenographic substances.

4. A method of fixating a graft as in claim 3, wherein the one or more alloplastic substances include: porous fluorohydroxyapatitic (FHA); hydroxyapatite-calcium phosphate; calcium phosphate (CaP); betatricalcium phosphate (b-TCP); hydroxyapatite ceramic; HTR (hard tissue replacement polymer); bioglass; tricalcium phosphate; ceramic; and titanium.

5. A method of fixating a graft as in claim 3, wherein the at least one xenographic substance is selected from the group comprising: bovine; coral; and algae.

6. A method of fixating a graft as in claim 1, wherein said selected one of a plurality of cylindrical grafts has a diameter of between 3 mm and 20 mm.

7. A method of fixating a graft as in claim 1, wherein said selected one of a plurality of cylindrical grafts has a height of between 2 mm and 10 mm.

8. A bone graft for fixating to an area of deficient bone in the jaws comprising:
a cylindrical body having a first end, a second end, a length defined between the first and second ends, and a uniform transverse cross-sectional diameter along the length; and
said cylindrical body formed from one or more materials selected from the group comprising:
allogeneic bone;
alloplasts; and
xenographs.

9. The graft as in claim 8, wherein alloplasts include:
porous fluorohydroxyapatitic (FHA);
hydroxyapatite-calcium phosphate;
calcium phosphate (CaP);
betatricalcium phosphate (b-TCP);
hydroxyapatite ceramic;
HTR (hard tissue replacement polymer);
bioglass;
tricalcium phosphate;
ceramic; and
titanium.

10. The graft as in claim 8, wherein the xenographs include:
bovine;
coral; and
algae.
